(19) United States
(12) Patent Application Publication
(10) Pub. No.: US 2023/0048490 A1
(43) Pub. Date: Feb. 16, 2023

(54) ANTENNA DEVICE FOR MEASURING BIOMETRIC INFORMATION BY USING LEAKY WAVE

(71) Applicant: SB Solutions Inc., Ulsan (KR)

(72) Inventors: Seungup Seo, Ulsan (KR); Namhwan Sung, Ulsan (KR); Hae Dong Lee, Ulsan (KR); Seong Mun Kim, Ulsan (KR); Ji Woong Song, Ulsan (KR); Jagannath Malik, Ulsan (KR)

(73) Assignee: SB Solutions Inc., Ulsan (KR)

(21) Appl. No.: 17/535,404

(22) Filed: Nov. 24, 2021

(30) Foreign Application Priority Data
Aug. 13, 2021 (KR) 10-2021-0107641

(51) Int. Cl. A61B 5/145; H01Q 13/20; H01Q 1/38; A61B 5/05

(52) U.S. Cl. CPC A61B 5/14532; H01Q 13/20; H01Q 1/38; A61B 5/05; A61B 5/1451

(57) ABSTRACT

Disclosed is an antenna device for measuring biometric information by using a leaky wave. The antenna device according to an embodiment may include an antenna main body formed to surround at least some portion of the body having a target analyte. The antenna main body may include a plurality of transmission-side slots and a plurality of reception-side slots formed on a surface surrounding the body portion. An electromagnetic wave is excited inside the antenna main body may be radiated to an inside of the body portion through at least one of the plurality of transmission-side slots. Information on an analyte within the body portion may be sensed based on a frequency of an electromagnetic wave received through at least one of the plurality of reception-side slots via the body portion.

### CROSS-REFERENCES TO RELATED APPLICATIONS

This application is based on and claims priority under 35 U.S.C. 119 to Korean Patent Application No. 10-2021-0107641, filed on Aug. 13, 2021 in the Korean intellectual property office, the disclosures of which are herein incorporated by reference in their entireties.

### TECHNICAL FIELD

The following description relates to an antenna device for measuring biometric information by using a leaky wave.

### BACKGROUND OF THE DISCLOSURE

Cases in which adult-onset diseases, such as diabetes, hyperlipidemia and thrombosis, are increased continue to increase. Such diseases need to be periodically measured using various bio sensors because it is important to continuously monitor and manage the diseases. A common type of a bio sensor is a method of injecting, into a test strip, blood gathered from a finger and then quantizing an output signal by using an electrochemical method or a photometry method. Such an approach method causes a user a lot of pain because blood needs to be gathered every time. For example, in order to manage diabetes of hundreds of millions of people worldwide, the most basic thing is to measure blood glucose. Accordingly, a blood glucose measuring device is an important diagnostic device inevitably necessary for a diabetic. Various blood glucose measuring devices are recently developed, but the most frequently used method is a method of gathering blood by pricking a finger and then directly measuring a concentration of glucose within the blood. An invasive test includes a method of measuring blood glucose through the recognition of an external reader after measuring the blood glucose for a given time by penetrating an invasive sensor into the skin.

In contrast, a non-invasive test includes a method using a light-emitting diode (LED)-photo diode (PD), etc. However, the non-invasive test has low accuracy due to an environmental factor, such as sweat or a temperature, an alien substance, etc. because the LED-PD is attached to the skin.

The aforementioned information is to merely help understanding, and may include contents which do not form a part of a conventional technology and may not include contents which may be presented to those skilled in the art through a conventional technology.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Embodiments of the present disclosure provide an antenna device for measuring biometric information by using a leaky wave.

In an embodiment, there is provided a leaky wave antenna device including an antenna main body formed to surround at least some of a body portion of a target analyte, wherein the antenna main body includes a plurality of transmission-side slots and a plurality of reception-side slots formed on a surface surrounding the body portion, an electromagnetic wave is excited inside the antenna main body is radiated to the inside of the body portion through at least one of the plurality of transmission-side slots, and information on an analyte within the body portion is sensed based on a frequency of an electromagnetic wave received through at least one of the plurality of reception-side slots via the body portion.

According to an aspect, the radiated electromagnetic wave may be different in the direction of a radiated beam depending on a frequency.

According to another aspect, a path of the radiated electromagnetic wave may be changed depending on a dielectric constant within the body portion.

According to still another aspect, a dielectric constant within the body portion may be measured based on a frequency of an electromagnetic wave forming the strongest channel between a slot from which the electromagnetic wave is radiated and a slot to which the electromagnetic wave is received.

According to still another aspect, the plurality of transmission-side slots and the plurality of reception-side slots may be formed in the antenna main body in a direction perpendicular to a direction in which the antenna main body surrounds the body portion.

According to still another aspect, the plurality of transmission-side slots or the plurality of reception-side slots may be formed so that the lengths of a (the “a” is a natural number) slots in the middle thereof are identical, the lengths of b (the “b” is a natural number) slots on the left side thereof relatively become smaller toward the left side, and the lengths of c (the “c” is a natural number) slots on the right side thereof relatively becomes smaller toward the right side. The number of plurality of transmission-side slots or plurality of reception-side slots may be determined as the sum of the a, the b and the c.

In an embodiment, there is provided a leaky wave antenna device including a flexible printed circuit board formed to surround at least some of a body portion of a target analyte, wherein a composite right/left-handed (CRLH) transmission wire is printed on a surface of the flexible printed circuit board surrounding the body portion, an electromagnetic wave having directivity is radiated to the inside of the body portion through a first portion of the CRLH transmission wire, and information on an analyte within the body portion is sensed based on a frequency of an electromagnetic wave sensed through a second portion of the CRLH transmission wire.

According to embodiments, there can be provided the antenna device for measuring biometric information by using a leaky wave.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a block diagram illustrating an example of a biometric information measuring apparatus according to an embodiment of the present disclosure.

FIG. 2 is an exemplary diagram illustrating three modes of the biometric information measuring apparatus according to an embodiment of the present disclosure.

FIG. 3 is a diagram illustrating an example of a leaky wave antenna according to an embodiment of the present disclosure.

FIG. 4 is a diagram illustrating an example in which a path of an electromagnetic wave is changed depending on a dielectric constant in an embodiment of the present disclosure.

FIG. 5 is a diagram illustrating an example of the concept of a leaky wave antenna capable of adjusting a beam direction in an embodiment of the present disclosure.

FIG. 6 is a diagram illustrating an implementation example of a leaky wave antenna according to an embodiment of the present disclosure.

FIGS. 7 to 10 are graphs illustrating the results of experiments of a leaky wave antenna implemented according to an embodiment of the present disclosure.

FIG. 11 is a diagram illustrating another implementation example of a leaky wave antenna according to an embodiment of the present disclosure.

FIG. 12 is a diagram illustrating still another implementation example of a leaky wave antenna according to an embodiment of the present disclosure.

FIG. 13 is a diagram illustrating examples of radiation patterns of the leaky wave antenna according to an embodiment of the present disclosure.

FIG. 14 is a diagram illustrating an example of a leaky wave antenna surrounding a body tissue in an embodiment of the present disclosure.

FIG. 15 is a graph illustrating that a scattering parameter is changed as a dielectric constant of a body tissue is changed in an embodiment of the present disclosure.

FIG. 16 is a diagram illustrating an example of a radiation pattern for an electromagnetic wave of a frequency of 10.8 GHz in an embodiment of the present disclosure.

FIGS. 17 and 18 are diagrams illustrating a structure and concept of a leaky wave antenna in an embodiment of the present disclosure.

FIG. 19 is a diagram illustrating an example of a patch antenna implemented to surround an arm of a person.

FIG. 20 is a diagram illustrating an example of a leaky wave antenna implemented to surround an arm of a person in an embodiment of the present disclosure.

FIG. 21 is a diagram illustrating an example of a leaky wave antenna using a composite right/left-handed (CRLH) transmission wire in an embodiment of the present disclosure.

FIG. 22 is a distribution diagram of the leaky wave antenna according to an embodiment of the present disclosure.

FIG. 23 is a graph illustrating examples of radiation patterns of the leaky wave antenna according to an embodiment of the present disclosure.

### DETAILED DESCRIPTION

While illustrative embodiments have been illustrated and described, it will be appreciated that various changes can be made therein without departing from the spirit and scope of the disclosure.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the claims of the application.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessively formal unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the subject matter of the embodiment unnecessarily vague.

Furthermore, in describing elements of an embodiment, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

According to an embodiment, there is provided a technique relating to an in-body bio sensor capable of semi-permanently measuring blood glucose. The in-body bio sensor may also be called an invasive type bio sensor, an insertion type bio sensor, or an implant type bio sensor. The in-body bio sensor may be a sensor for sensing a target analyte by using an electromagnetic wave. For example, the in-body bio sensor may measure biometric information associated with a target analyte. Hereinafter, the target analyte is a material associated with a living body, and may also be called a living body material or an analyte. For reference, in this specification, the target analyte is chiefly described as blood glucose, but the present disclosure is not limited thereto. The biometric information is information related to a bio component of a target, and may include a concentration of a target analyte or a numerical value, for example. If a target analyte is blood glucose, biometric information may include a blood glucose numerical value.

The in-body bio sensor may measure a bio parameter (hereinafter referred to as a “parameter”) associated with a bio component, and may determine biometric information from the measured parameter. In this specification, a parameter may indicate a circuit network parameter used to interpret a bio sensor and/or a bio sensing system, and is described by chiefly taking a scattering parameter as an example, for convenience of description, but the present disclosure is not limited thereto. For example, an admittance parameter, an impedance parameter, a hybrid parameter, a transmission parameter, etc. may be used as the parameter. A permeability coefficient and a reflection coefficient may be used as the scattering parameter. For reference, a resonant frequency calculated from a scattering parameter may be related to a concentration of a target analyte. The in-body bio sensor may predict blood glucose by sensing a change in the permeability coefficient and/or the reflection coefficient.

The in-body bio sensor may include a resonator assembly (e.g., an antenna). Hereinafter, an example in which the resonator assembly is an antenna is chiefly described. A resonant frequency of an antenna may be represented as a capacitance component and an inductance component as in Equation 1.

$$f = \frac{1}{2 π \sqrt{L C}} \qquad [ Equation 1 ]$$

In Equation 1,ƒmay indicate the resonant frequency of the antenna included in the in-body bio sensor using an electromagnetic wave. L may indicate inductance of the antenna. C may indicate capacitance of the antenna. The capacitance C of the antenna may be proportional to a relative dielectric constant εᵣ as in Equation 2 below.

C∝εᵣ [Equation 2]

The relative dielectric constant εᵣ of the antenna may be influenced by a concentration of a surrounding target analyte. For example, when an electromagnetic wave passes through a material having a given dielectric constant, amplitude and a phase of the electromagnetic wave may be changed due to the reflection and scattering of the electromagnetic wave. The relative dielectric constant εᵣ may also vary because a degree of the reflection and/or scattering of the electromagnetic wave is different depending on a concentration of a target analyte around the in-body bio sensor. It may be interpreted that bio capacitance is formed between the in-body bio sensor and the target analyte due to a fringing field attributable to the electromagnetic wave radiated by the in-body bio sensor including an antenna. A resonant frequency of the antenna also varies because the relative dielectric constant εᵣ of the antenna varies depending on a change in the analyte concentration. In other words, the analyte concentration may correspond to the resonant frequency.

The in-body bio sensor according to an embodiment may radiate an electromagnetic wave while sweeping a frequency, and may measure a scattering parameter according to the radiated electromagnetic wave. The in-body bio sensor may determine a resonant frequency from the measured scattering parameter, and may estimate a blood glucose numerical value corresponding to the determined resonant frequency. The in-body bio sensor may be inserted into a subcutaneous layer, and may predict blood glucose diffused from a blood vessel to an interstitial fluid. The in-body bio sensor may estimate biometric information by determining a frequency shift degree of a resonant frequency. In order to more accurately measure the resonant frequency, a quality factor may be maximized. Hereinafter, an antenna structure having an improved quality factor in an antenna device used in a bio sensor using an electromagnetic wave is described.

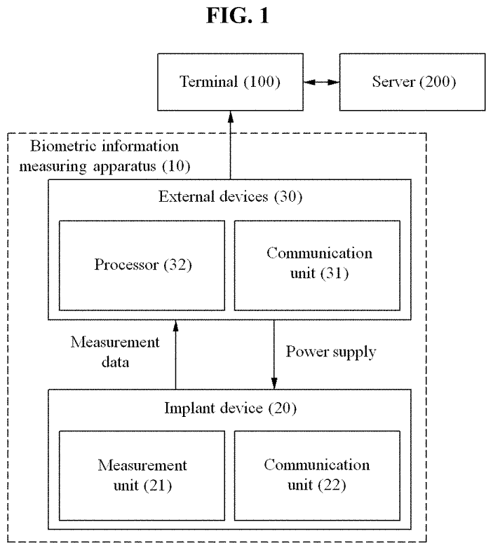

FIG. 1 is a block diagram illustrating an example of a biometric information measuring apparatus according to an embodiment of the present disclosure. The biometric information measuring apparatus 10 according to the present embodiment may include an implant device 20 inserted into the body of a target analyte whose biometric information (e.g., an analyte concentration, such as blood glucose or oxygen saturation) is to be measured and an external devices 30 disposed in the exterior of the target analyte at a location corresponding to a location of the implant device 20. The target analyte may be a human being or an animal. In this case, the implant device 20 may correspond to the aforementioned in-body bio sensor.

The external device 30 is a sensor attached to the outside of the body of the target analyte or worn by the target analyte, and may be fixed to the exterior of the target analyte by using various methods, such as a bending method and an adhesive method. The external device 30 may include a communication unit 31, and the external devices 30 may be paired through the communication units 31 or may provide biometric information to a preset terminal 100.

According to an embodiment, the external device 30 may also provide biometric information itself to the terminal 100, may perform a variety of types of analysis on the biometric information, and may provide the terminal 100 with the results of the analysis, a warning, etc. If the external device 30 provides biometric information itself to the terminal 100, the terminal 100 may perform a variety of types of analysis on the biometric information. Means for analyzing such biometric information may be easily selected by a practicer.

Furthermore, the external devices 30 can secure measurement accuracy and measurement continuity by blocking a performance change attributable to an external environment. The external devices 30 can improve accuracy by securing complementary data with the implant device 20.

The implant device 20 may be inserted into the body of a target analyte. For example, the implant device 20 does not directly come into contact with blood or is not disposed within a blood vessel, but may be disposed in an area other than a blood vessel at a given depth from the skin of a target. In other words, the implant device 20 is preferably disposed in a hypodermic area between the skin and the blood vessel.

The implant device 20 may radiate an electromagnetic wave having a specific frequency, and may measure a concentration of an analyte by measuring a signal reflected by the analyte around a sensor. For example, if blood glucose is to be measured, the implant device 20 may radiate an electromagnetic wave having a specific frequency, and may measure biometric information, such as blood glucose, by measuring a signal reflected by an analyte, such as glucose around a sensor.

The external devices 30 may be disposed in the exterior of a target analyte at a location corresponding to a location where the implant device 20 is disposed, supply power to the implant device 20, and may receive measurement data (e.g., the aforementioned biometric information) measured by the implant device 20.

When a concentration (e.g., a blood glucose numerical value) of a target analyte within a blood vessel of the target analyte is changed, a concentration of the analyte in a hypodermic area may be changed. In this case, a dielectric constant in the hypodermic area may be changed in response to a change in the concentration of the analyte. At this time, a resonant frequency in a measurement unit 21 of the implant device 20 may be changed in response to a change in the dielectric constant of a surrounding hypodermic area. For example, the measurement unit 21 may include a conducting wire having a specific pattern and a feeder line. In this case, when the dielectric constant of the surrounding hypodermic area is changed, a resonant frequency attributable to the specific pattern and the feeder line may also be changed because capacitance of the measurement unit 21 is changed. An analyte concentration under the skin is changed in proportion to an analyte concentration of a neighbor blood vessel. Accordingly, the biometric information measuring apparatus 10 may finally calculate biometric information, such as an analyte concentration, by using a resonant frequency corresponding to a change in the dielectric constant under the skin.

As an embodiment, the biometric information measuring apparatus 10 may calculate a corresponding relative dielectric constant by using a frequency (e.g., a resonant frequency) at a point at which the size of a scattering parameter is the smallest or greatest.

As an embodiment, the measurement unit 21 of the implant device 20 may be constructed in the form of a resonant device. The implant device 20 may generate a signal by sweeping a frequency within a pre-designated frequency band and inject the generated signal into the resonant device. At this time, the external devices 30 may measure a scattering parameter with respect to the resonant device to which a signal having a varying resonant frequency is supplied.

A communication unit 22 of the implant device 20 may transmit, to the external devices 30, data measured by the measurement unit 21. The communication unit 22 may receive, from the external device 30, power for generating a signal supplied to the measurement unit 21 by using a wireless power transmission method.

The external devices 30 may include a processor 32 and the communication unit 31. The communication unit 31 may receive measurement data (e.g., a scattering parameter or a degree of a change in the resonant frequency) measured by the implant device 20. In this case, the processor 32 of the external devices 30 may determine an analyte concentration based on the measurement data received from the implant device 20. According to an embodiment, the analyte concentration may be directly determined by the external devices 30, but may be determined by the terminal 100 that receives the measurement data from the external devices 30.

As an embodiment, a lookup table (LUT) in which measurement data (e.g., a scattering parameter and/or a degree of a change in the resonant frequency) and analyte concentrations are previously mapped may be stored in the external devices 30. The processor 32 may load an analyte concentration based on the LUT.

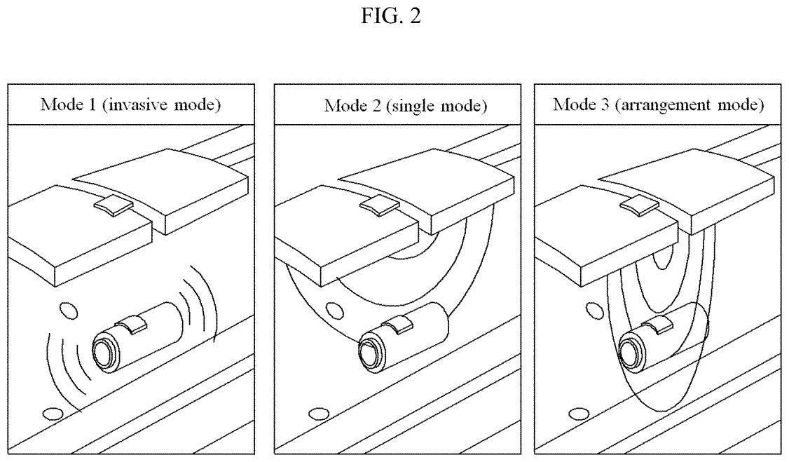

FIG. 2 is an exemplary diagram illustrating three modes of the biometric information measuring apparatus according to an embodiment of the present disclosure. The biometric information measuring apparatus 10 according to the present embodiment may operate in the three modes. The three modes may be independently performed or may be alternately performed at given time intervals.

<Mode 1: invasive mode>

In Mode 1, the biometric information measuring apparatus 10 may directly measure an analyte diffused from a blood vessel of a target to an interstitial fluid within a tissue. For example, an IC chip as the measurement unit 21 of the implant device 20 may radiate an electromagnetic wave having a specific frequency to an analyte, such as glucose around the implant device 20, and may measure a signal reflected and returned from the analyte. Furthermore, the implant device 20 may output a waveform (e.g., a sine wave) of a resonant frequency that varies over time. When a reflection signal according to the frequency in a specific time is detected, the implant device 20 may generate measurement data for biometric information corresponding to the frequency.

<Mode 2: single mode>

In Mode 2, at least one external device 30 is provided. Preferably, two external devices 30 may be provided. In this case, in Mode 2, the external devices 30 may include a first external device and a second external device disposed at a given interval.

The biometric information measuring apparatus 10 is coupled to the first external device and the second external device disposed at a given intervals, may measure an electromagnetic wave according to a change in an analyte concentration within an interstitial fluid on the outer surface of the skin of a target analyte, and may calibrate a measured value based on measurement data of biometric information of the implant device 20 along with the measured electromagnetic wave. The biometric information measuring apparatus 10 can improve the accuracy of measurement of an analyte concentration by calibrating a measured value through such a multi-mode.

<Mode 3: arrangement mode>

In Mode 1, the implant device 20 of the biometric information measuring apparatus 10 radiates an electromagnetic wave to an analyte around the implant device 20 and measures a signal reflected and returned from the analyte.

In contrast, in Mode 3, the biometric information measuring apparatus 10 radiates an electromagnetic wave that reaches even a depth of a blood vessel of a target analyte, and generates measurement data for biometric information (as an analyte concentration, for example, a blood glucose numerical value) based on a signal reflected and returned from the analyte within the blood vessel.

In general, when an analyte concentration within a blood vessel is changed, the analyte concentration in the hypodermic area may be changed. A dielectric constant in the hypodermic area is changed in response to a change in the analyte concentration.

In Modes 1 and 2, measurement data for biometric information is generated by performing measurement on such a hypodermic area. For this reason, there may be a difference between an analyte concentration within an actual blood vessel and an analyte concentration within a hypodermic area.

Accordingly, the biometric information measuring apparatus 10 can solve a time delay problem with an analyte concentration in a way to obtain measurement data for biometric information within an actual blood vessel by executing an operation, such as Mode 3. Furthermore, a problem which may occur in a target analyte during the time delay can be rapidly checked in advance because a sudden change in the analyte concentration of the target analyte can be measured by Mode 3.

Furthermore, the biometric information measuring apparatus 10 according to an embodiment can measure biometric information more accurately in a way to calibrate a value of an analyte concentration by simultaneously operating two or more of the three modes. For example, the biometric information measuring apparatus 10 according to the present embodiment can secure accuracy in a way to secure the diversity of data by simultaneously using Modes 1 and 2 of the implant device 20, and can improve the accuracy of measurement of biometric information through a repetition test.

Furthermore, the biometric information measuring apparatus 10 according to an embodiment can solve the time delay problem, that is, a problem with conventional interstitial fluid sensors for measuring biometric information, by improving a penetration depth of an electromagnetic wave radiated using Mode 3 and monitoring a change in the analyte concentration within a blood vessel in real time.

Furthermore, as described above, the diversity of data can be secured by using a plurality of sensors and a plurality of modes together. The accuracy of a method of predicting biometric information can be improved and a reappearance issue can be solved by adjusting a calibration cycle.

As an embodiment, the biometric information measuring apparatus 10 may predict an analyte concentration by associating, with a Bayesian filter-based algorithm, measurement data of another sensor (e.g., an environment sensor, a temperature sensor or a humidity sensor) along with measurement data measured by Modes 1, 2, and 3.

Furthermore, the biometric information measuring apparatus 10 according to an embodiment may simultaneously use Modes 1 and 2 in order to secure the reappearance of measurement of a dielectric constant, and may perform re-measurement when analyte concentrations measured based on measurement data of Mode 1 and measurement data of Mode 2 are not the same, or may measure an analyte concentration through blood-gathering, may input the measured analyte concentration, and may perform calibration.

As described above, the biometric information measuring apparatus 10 may perform mutual verification on the results of measurement when values of analyte concentrations measured in multiple modes are the same based on a plurality of measurement data obtained in the multiple modes, and may request to measure an analyte concentration of a target analyte through blood-gathering only when values of analyte concentrations measured in the multiples modes are different in order to reduce the number of times of blood-gathering for the target analyte.

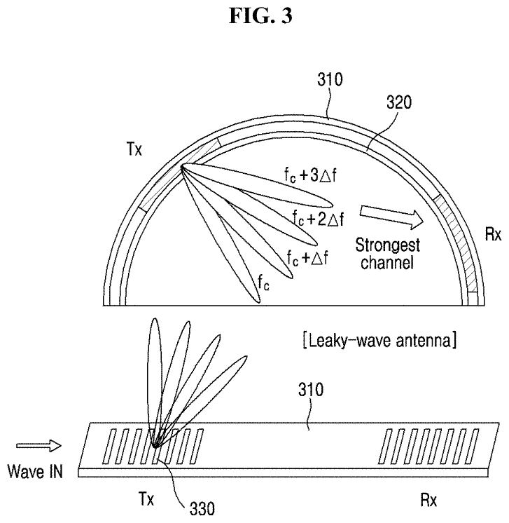

FIG. 3 is a diagram illustrating an example of a leaky wave antenna according to an embodiment of the present disclosure. A leaky wave antenna 310 according to the embodiment of FIG. 3 may be implemented in a band form. The leaky wave antenna may be disposed on a bent skin surface 320 of a target analyte, and may radiate, into the body of the target analyte, a magnetic field having directivity. For example, the leaky wave antenna 310 may be an example of an antenna included in the external devices 30 for Mode 2 (the single mode) and/or Mode 3 (the arrangement mode) described with reference to FIG. 2.

When an electromagnetic wave is excited inside the leaky wave antenna 310 made of a dielectric, the electromagnetic wave may pass through the inside of the leaky wave antenna 310, and may be then radiated to the outside of the leaky wave antenna 310 through a slot formed in the antenna main body of the leaky wave antenna 310. The antenna main body may include a plurality of transmission-side (Tx) slots and a plurality of reception-side (Rx) slots formed on a surface that surrounds a body portion of a target analyte. In this case, a direction in which the electromagnetic wave is radiated from the slot may vary depending on a frequency of the electromagnetic wave. The embodiment of FIG. 3 illustrates an example in which electromagnetic waves are radiated in different directions with respect to four frequencies “f_{c}”, “f_{c}+Δf”, “f_{c}+2Δf” and “f_{c}+3Δf” through a specific slot 330 of the plurality of Tx slots.

Furthermore, a path of the electromagnetic wave may be changed depending on a dielectric constant within the body of the target analyte. In this case, the same link may be formed between the transmission side Tx and the reception side Rx in the same dielectric constant. In this case, the same link may mean that an electromagnetic wave of the same frequency always forms the strongest channel between the transmission side Tx and the reception side Rx in the same dielectric constant. In this case, a dielectric constant may be measured through a frequency of the electromagnetic wave that forms the strongest channel between the transmission side Tx and the reception side Rx by designing the leaky wave antenna 310 so that a beam direction of the electromagnetic wave is different for each frequency. If a dielectric constant within the body of a target analyte is measured as described above, biometric information within the body may be obtained based on the measured dielectric constant.

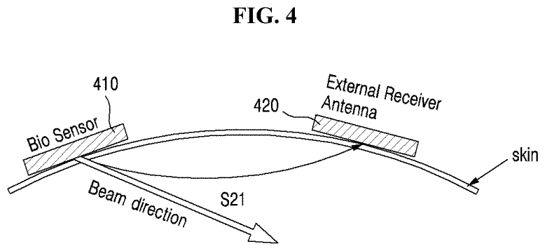

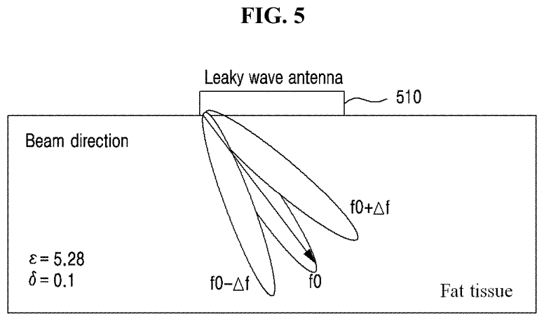

FIG. 4 is a diagram illustrating an example in which a path of an electromagnetic wave is changed depending on a dielectric constant in an embodiment of the present disclosure. FIG. 5 is a diagram illustrating an example of the concept of a leaky wave antenna capable of adjusting a beam direction in an embodiment of the present disclosure. FIG. 4 illustrates an example in which the path of a beam of an electromagnetic wave radiated in a specific direction through a bio sensor 410 disposed on the skin is changed depending on a dielectric constant and the beam reaches an external receiver antenna 420. In this case, the external receiver antenna 420 may be used as a biochemical sensor because a resonance frequency is influenced by a dielectric constant of a material. For example, the external receiver antenna 420 in FIG. 4 may obtain the results of the bio sensor through the results of a scattering parameter S21. In this case, FIG. 5 illustrates that the angle of a beam steered into the body of a target analyte may be controlled using a leaky wave antenna 510. In this case, a cost can be reduced because a beam direction of the leaky wave antenna 510 can be adjusted based on a frequency through one port.

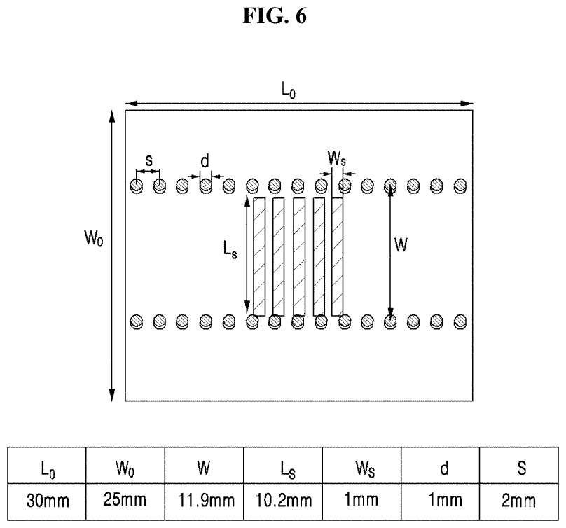

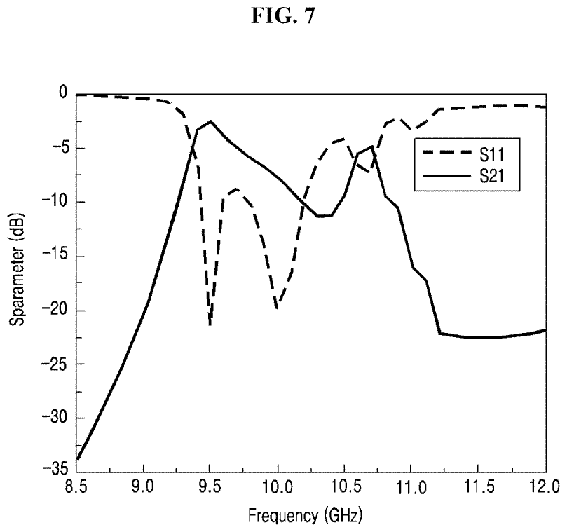

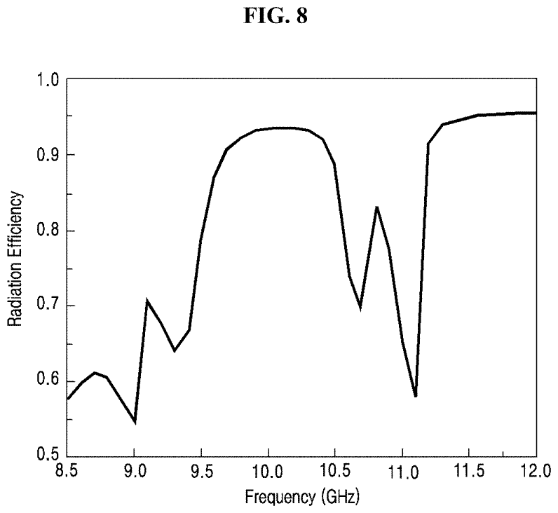

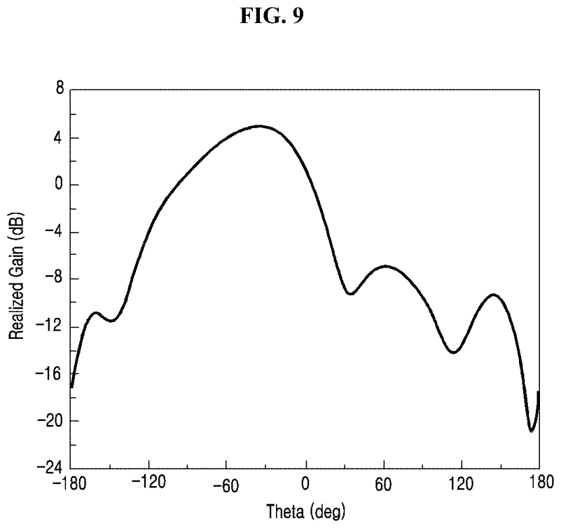

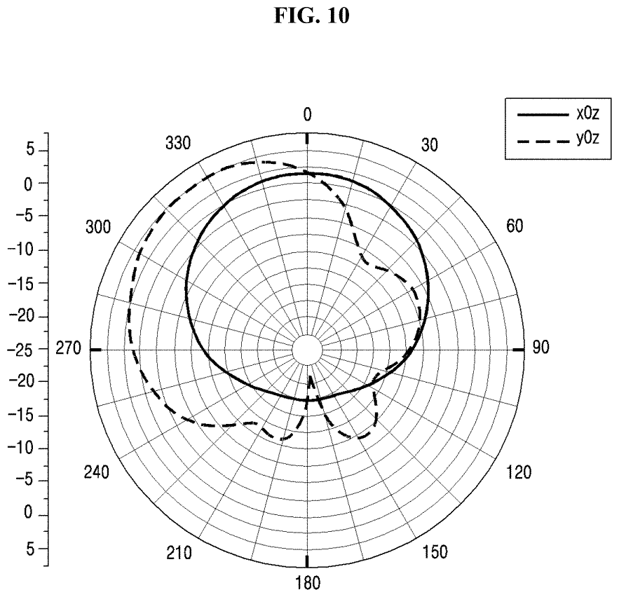

FIG. 6 is a diagram illustrating an implementation example of a leaky wave antenna according to an embodiment of the present disclosure. FIGS. 7 to 10 are graphs illustrating the results of experiments of a leaky wave antenna implemented according to an embodiment of the present disclosure. The graph of FIG. 7 illustrates scattering parameters according to frequencies. The graph of FIG. 8 illustrates radiation efficiency according to frequencies. The graph of FIG. 9 illustrates realized gains (losses) according to theta waves. FIG. 10 illustrates directivity in which a beam is formed for ach frequency. FIG. 10 illustrates that a radiated beam may have directivity depending on a frequency. The leaky wave antenna according to the present embodiment includes a lateral slot. A beam may be radiated to the outside of the leaky wave antenna through the slot with radiation efficiency 0.9 in a frequency of 10 GHz.

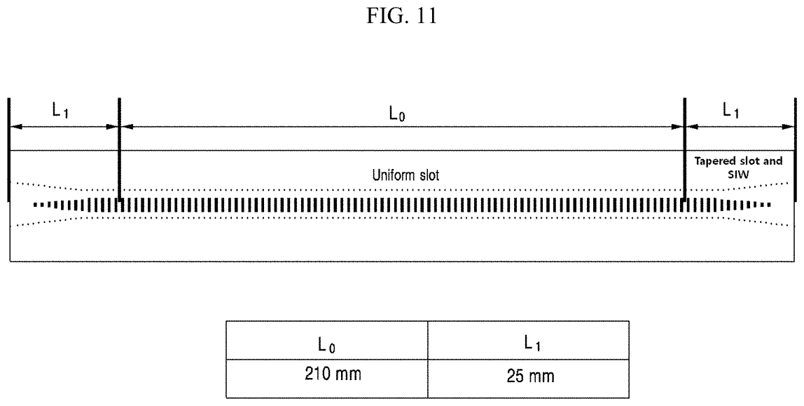

FIG. 11 is a diagram illustrating another implementation example of a leaky wave antenna according to an embodiment of the present disclosure. In order to achieve a high gain and a narrower beam width, higher L₀ may be set. Furthermore, a bandwidth can be improved through an excellent reflection loss S₁₁ by applying a taper slot and a substrate integrated waveguide (SIW) to both sides of the leaky wave antenna.

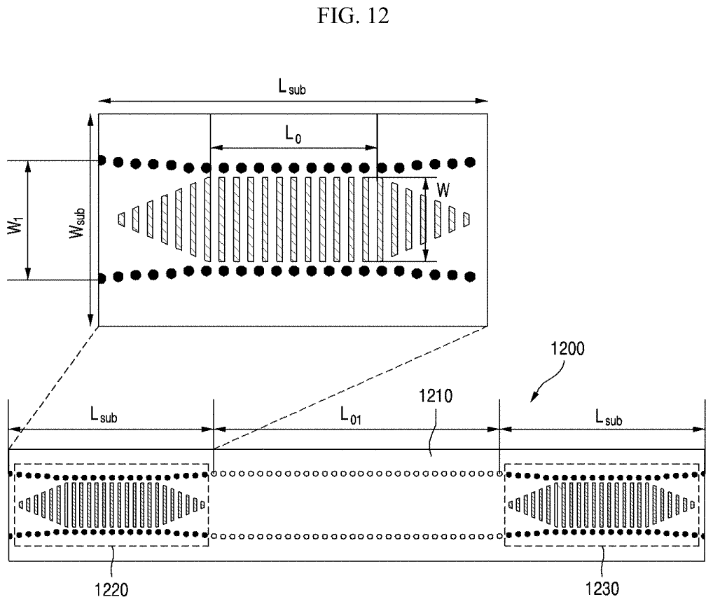

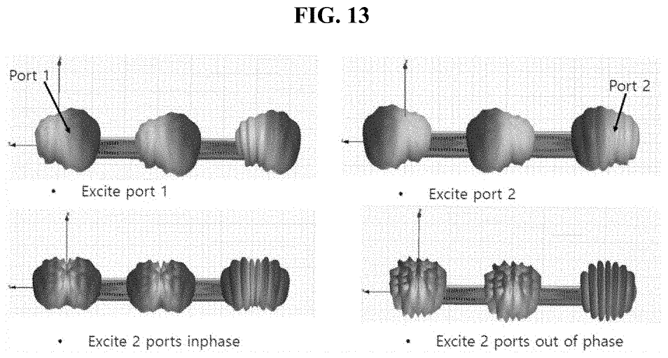

FIG. 12 is a diagram illustrating still another implementation example of a leaky wave antenna according to an embodiment of the present disclosure. A leaky wave antenna 1200 according to the present embodiment may include an antenna main body 1210 formed to surround at least some of a body portion of a target analyte. In this case, the antenna main body 1210 may include a plurality of Tx slots and a plurality of Rx slots formed on a surface that surrounds the body portion. a first dotted box 1220 may indicate the plurality of Tx slots, and a second dotted box 1230 may indicate the plurality of Rx slots. The plurality of Tx slots or the plurality of Rx slots may be formed so that the lengths of a (“a” is a natural number) slots in the middle thereof are the same, the lengths of b (“b” is a natural number) slots on the left side thereof relatively become smaller toward the left side, and the lengths of c (“c” is a natural number) slots on the right side thereof relatively becomes smaller toward the right side. In this case, the number of plurality of Tx slots or plurality of Rx slots may be determined as a +b+c. The leaky wave antenna 1200 according to the present embodiment may radiate, to the outside of the leaky wave antenna 1200, most of power having scattering parameters S₁₁ and S₂₁ in a frequency of −10 dB or less. FIG. 13 is a diagram illustrating examples of radiation patterns of the leaky wave antenna according to an embodiment of the present disclosure. FIG. 13 illustrates that when two ports are used on both sides of the leaky wave antenna, respectively, a radiation pattern may be different depending on through which port an electromagnetic wave is excited, and whether in-phase electromagnetic waves will be excited through the two ports or electromagnetic waves out of phase will be excited through the two ports when electromagnetic waves are excited through the two ports.

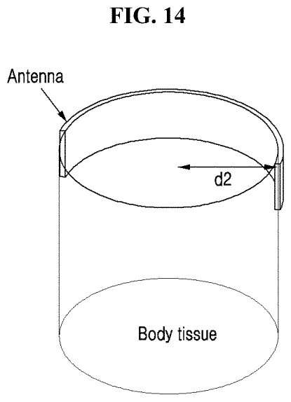

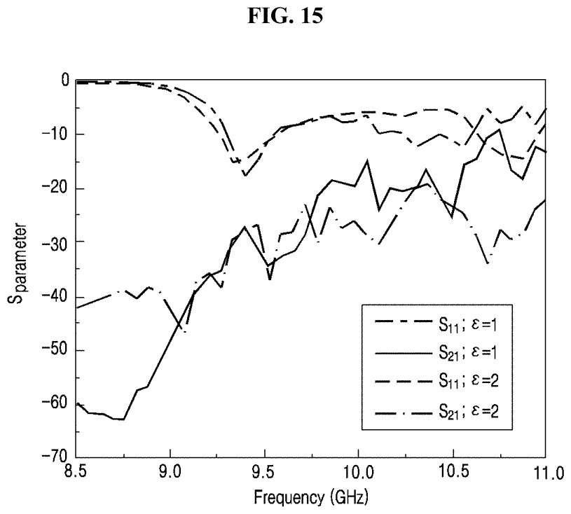

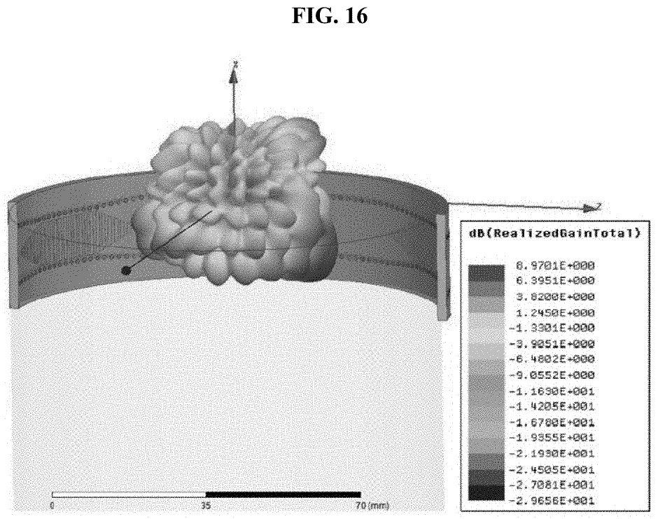

FIG. 14 is a diagram illustrating an example of a leaky wave antenna surrounding a body tissue in an embodiment of the present disclosure. FIG. 15 is a graph illustrating that a scattering parameter is changed as a dielectric constant of a body tissue is changed in an embodiment of the present disclosure. FIG. 16 is a diagram illustrating an example of a radiation pattern for an electromagnetic wave of a frequency of 10.8 GHz in an embodiment of the present disclosure.

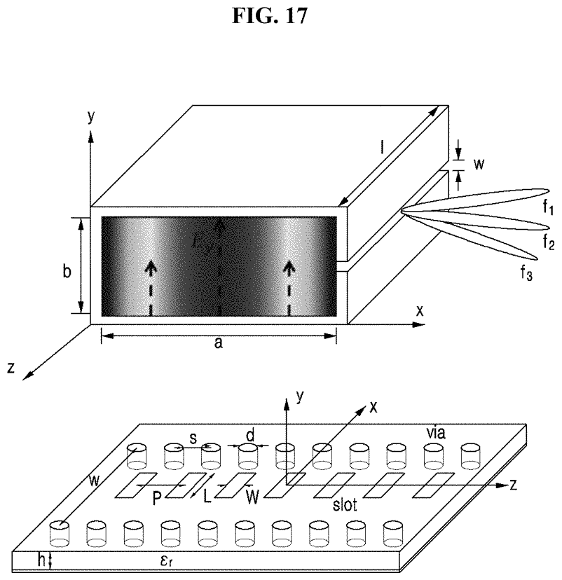

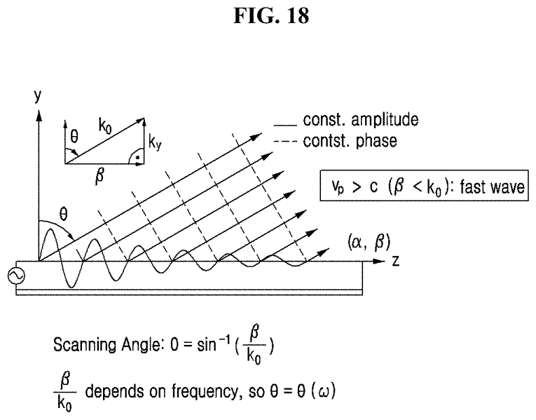

FIGS. 17 and 18 are diagrams illustrating a structure and concept of a leaky wave antenna in an embodiment of the present disclosure. The leaky wave antenna based on a traveling wave may perform a beam steering ability according to a frequency with high directivity through a simple structure. If slots are disposed in a waveguide structure, a wave may be radiated through a corresponding slot in a fast wave mode (i.e., a leaky wave mode).

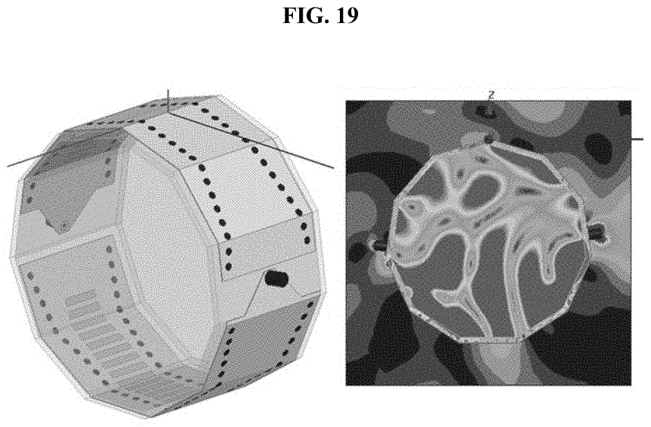

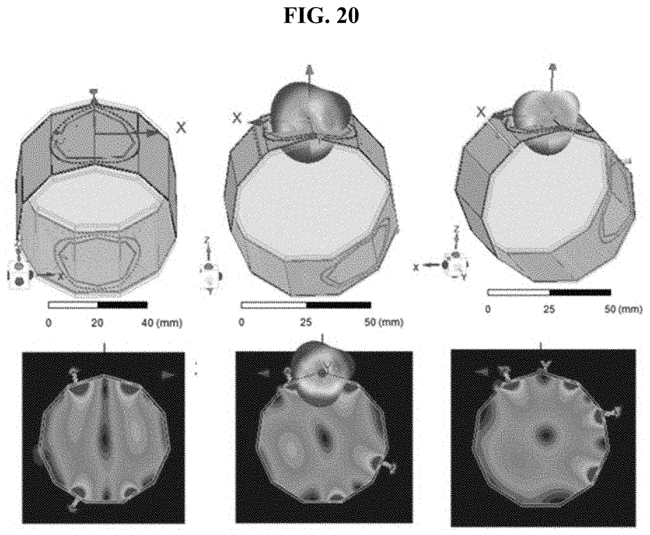

FIG. 19 is a diagram illustrating an example of a patch antenna implemented to surround an arm of a person. FIG. 20 is a diagram illustrating an example of a leaky wave antenna implemented to surround an arm of a person in an embodiment of the present disclosure. The strongest bonding strength between two antennas (i.e., a transmission side (Tx) and a reception side (Rx)) wound on the arm may be controlled by a combination of differences between a radiation direction and a location where the leaky wave antenna is disposed. In this case, as in FIG. 20, in the case of the leaky wave antenna, all combined fields between the two antennas may be concentrated on the body of a target analyte.

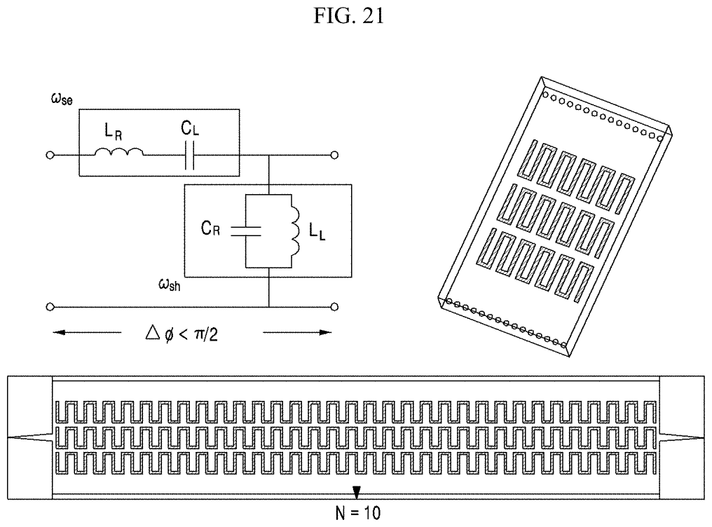

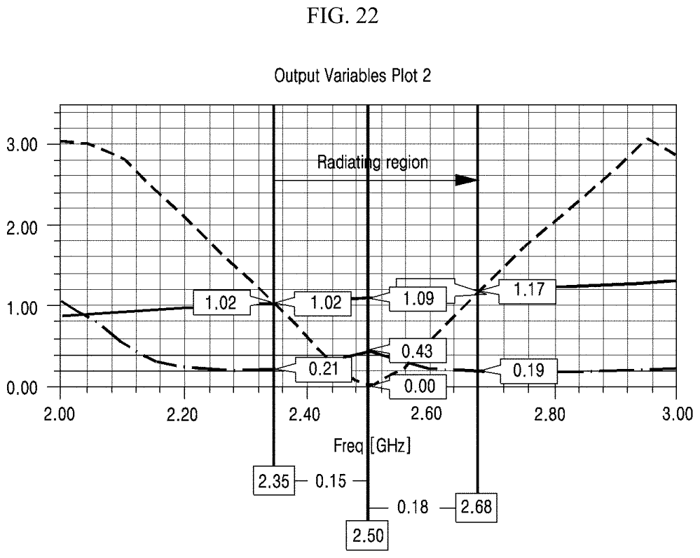

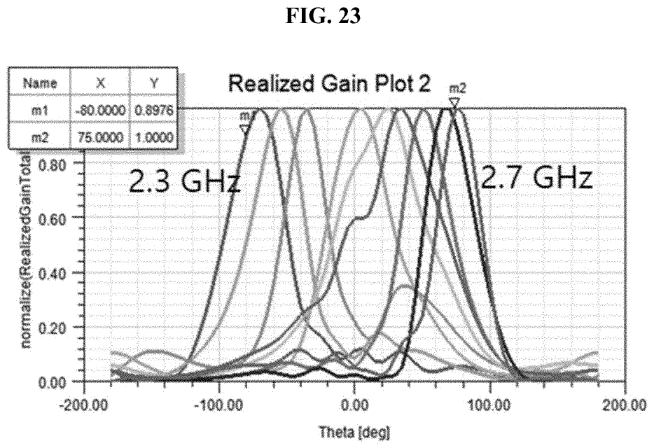

FIG. 21 is a diagram illustrating an example of a leaky wave antenna using a composite right/left-handed (CRLH) transmission wire in an embodiment of the present disclosure. FIG. 22 is a distribution diagram of the leaky wave antenna according to an embodiment of the present disclosure. FIG. 23 is a graph illustrating examples of radiation patterns of the leaky wave antenna according to an embodiment of the present disclosure. For example, the leaky wave antenna of FIG. 21 according to an embodiment is an antenna including a flexible printed circuit board having a fixed thickness of 0.22 mm. The size of a slot disposed in metal on the upper side may be adjusted in order to achieve a balance condition (L_{R}C_{L}=L_{L}C_{R}) for the CRLH transmission wire capable of providing a continuous beam scanning ability up to forward radiation at the back thereof. The CRLH transmission wire may be printed on the flexible printed circuit board. A beam in a preset direction may be formed with respect to an electromagnetic wave through the CRLH transmission wire. For example, the leaky wave antenna according to the present embodiment may radiate, into the inside of a body portion, an electromagnetic wave having directivity through a first portion of the CRLH transmission wire, and may sense information on an analyte within the body portion based on a frequency of the electromagnetic wave sensed through a second portion of the CRLH transmission wire. As described above, a path of the radiated electromagnetic wave may be changed depending on a dielectric constant within the body portion. In this case, the leaky wave antenna may measure the dielectric constant within the body portion based on the frequency of the electromagnetic wave that forms the strongest channel between the first portion from which the electromagnetic wave is radiated and the second portion to which the electromagnetic wave is received. As described above, the size of the slot of the CRLH transmission wire may be adjusted in order to achieve the balance condition based on capacitance and inductance of a capacitor and an inductor for supplying power to the CRLH transmission wire.

As described above, according to embodiments of the present disclosure, there can be provided the antenna device for measuring biometric information by using a leaky wave.

The aforementioned system or device or apparatus may be implemented as a hardware component or a combination of a hardware component and a software component. For example, the device and component described in the embodiments may be implemented using a processor, a controller, an arithmetic logic unit (ALU), a digital signal processor, a microcomputer, a field programmable gate array (FPGA), a programmable logic unit (PLU), a microprocessor or one or more general-purpose computers or special-purpose computers, such as any other device capable of executing or responding to an instruction. The processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary skill in the art may understand that the processing device may include a plurality of processing elements and/or a plurality of types of processing elements. For example, the processing device may include a plurality of processors or a single processor and a single controller. Furthermore, a different processing configuration, such as a parallel processor, is also possible.

Software may include a computer program, a code, an instruction or a combination of one or more of them and may configure a processing device so that the processing device operates as desired or may instruct the processing devices independently or collectively. The software and/or the data may be embodied in any type of machine, a component, a physical device, a computer storage medium or a device in order to be interpreted by the processor or to provide an instruction or data to the processing device. The software may be distributed to computer systems connected over a network and may be stored or executed in a distributed manner. The software and the data may be stored in one or more computer-readable recording media.

The method according to embodiments may be implemented in the form of a program instruction executable by various computer means and stored in a computer-readable medium. The computer-readable medium may include a program instruction, a data file, and a data structure solely or in combination. The medium may continue to store a program executable by a computer or may temporarily store the program for execution or download. Furthermore, the medium may be various recording means or storage means having a form in which one or a plurality of pieces of hardware has been combined. The medium is not limited to a medium directly connected to a computer system, but may be one distributed over a network. An example of the medium may be one configured to store program instructions, including magnetic media such as a hard disk, a floppy disk and a magnetic tape, optical media such as a CD-ROM and a DVD, magneto-optical media such as a floptical disk, a ROM, a RAM, and a flash memory. Furthermore, other examples of the medium may include an app store in which apps are distributed, a site in which other various pieces of software are supplied or distributed, and recording media and/or storage media managed in a server. Examples of the program instruction may include machine-language code, such as a code written by a compiler, and a high-level language code executable by a computer using an interpreter.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as the system, configuration, device, and circuit, are coupled or combined in a form different from that of the described method or replaced or substituted with other components or equivalents.

Accordingly, other implementations, other embodiments, and the equivalents of the claims fall within the scope of the claims.

1. A leaky wave antenna device comprising:
an antenna main body formed to surround at least some of a body portion, wherein the body portion contains a target analyte, and
a processor configured to predict a concentration of the target analyte by associating, with a Bayesian filter-based algorithm, measurement data of an another sensor, and information on an analyte sensed,
wherein the antenna main body comprises a plurality of transmission-side slots and a plurality of reception-side slots formed on a surface surrounding the body portion,
an electromagnetic wave is excited inside the antenna main body and radiated to an inside of the body portion through at least one of the plurality of transmission-side slots, and
a first port and a second port located on a first side of the leaky wave antenna device, configured to receive the electromagnetic wave,
a third port and a fourth port located on a second side of the leaky wave antenna device, configured to receive the electromagnetic wave after the electromagnetic wave passes through the first and second ports,
wherein when the electromagnetic wave passes through the first, second, third, and fourth port a radiation pattern determined by which port the electromagnetic wave is excited through is generated,
the information on an analyte within the body portion is sensed based on a frequency of an electromagnetic wave received through at least one of the plurality of reception-side slots via the body portion.

2. (canceled)

3. The leaky wave antenna device of claim 1, wherein a path of the radiated electromagnetic wave is changed depending on a dielectric constant within the body portion.

4. (canceled)

5. The leaky wave antenna device of claim 1, wherein the plurality of transmission-side slots and the plurality of reception-side slots are formed in the antenna main body in a direction perpendicular to a direction in which the antenna main body surrounds the body portion.

6. The leaky wave antenna device of claim 1, wherein:
the plurality of transmission-side slots or the plurality of reception-side slots are formed so that lengths of a (the “a” is a natural number) slots in a middle thereof are identical, lengths of b (the “b” is a natural number) slots on a left side thereof relatively become smaller toward the left side, and lengths of c (the “c” is a natural number) slots on a right side thereof relatively becomes smaller toward the right side, and
the number of plurality of transmission-side slots or plurality of reception-side slots is determined as a sum of the a, the band the c.

7. A leaky wave antenna device comprising:
a flexible printed circuit board formed to surround at least some of a body portion of a target analyte,
wherein a composite right/left-handed (CRLH) transmission wire is printed on a surface of the flexible printed circuit board surrounding the body portion,
an electromagnetic wave having directivity is radiated to an inside of the body portion through a first portion of the CRLH transmission wire, and
information on an analyte within the body portion is sensed based on a frequency of an electromagnetic wave sensed through a second portion of the CRLH transmission wire.

8. The leaky wave antenna device of claim 7, wherein a path of the radiated electromagnetic wave is changed depending on a dielectric constant within the body portion.

9. The leaky wave antenna device of claim 7, wherein a dielectric constant within the body portion is measured based on a frequency of an electromagnetic wave forming a strongest channel between the first portion from which the electromagnetic wave is radiated and the second portion to which the electromagnetic wave is received.

10. The leaky wave antenna device of claim 7, wherein a size of a slot of the CRLH transmission wire is adjusted in order to achieve a balance condition based on capacitance and inductance of a capacitor and an inductor for supplying power to the CRLH transmission wire.

11. The leaky wave antenna of claim 1, wherein the another sensor is selected from an environmental sensor, a temperature sensor, or a humidity sensor.
